(19) United States
(12) Patent Application Publication
(10) Pub. No.: US 2025/0382260 A1
(43) Pub. Date: Dec. 18, 2025

(54) POLYMERIC MATERIALS AND ADDITIVES THEREOF

(71) Applicant: Colormatrix Holdings, Inc., Avon Lake, OH (US)

(72) Inventors: Matthew JACKSON, Knowsley Merseyside (GB); Christopher STARKIE, Knowsley Merseyside (GB)

(21) Appl. No.: 18/878,770

(22) PCT Filed: Jun. 20, 2023
(86) PCT No.: PCT/IB2023/056362

§ 371 Date: Dec. 24, 2024

(30) Foreign Application Priority Data
Jun. 29, 2022 (GB) 2209528.5

(51) Int. Cl. C07C 237/40; C08J 3/22

(52) U.S. Cl. CPC C07C 237/40; C08J 3/226

(57) ABSTRACT

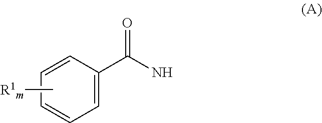

A formulation for decreasing aldehyde content in a polymeric material includes a compound which includes at least three moicties of formula (A) wherein each moiety (A) includes an amine moiety (—NH2) bonded ortho or meta to the amide moiety (—CONH); wherein each R1I independently represents a substituent and m is an integer from 0 to 4; and wherein the three moieties (A) are bonded, via their respective amide nitrogen atoms, to respective carbon atoms of a Main Fragment, wherein the Main Fragment includes carbon and hydrogen atoms only and is saturated.

This invention relates to polymeric materials and particularly, although not exclusively, relates to polymeric materials, such as polyesters, polyoxymethylenes (POMs), polyolefins, polyketones and polyurethanes, in which aldehyde may undesirably be associated, for example by virtue of being produced during manufacture of the polymeric materials, during downstream melt-processing of the polymeric materials and/or during use thereof. Said polymeric materials may also comprises recycled material which may be contaminated with aldehyde. This may particularly apply to recycled HDPE and/or PP.

Polyethylene terephthalate (PET) is used on a large scale for the manufacture of food packages such as bottles. Such bottles are widely utilised for packaging of beverages, such as carbonated soft drinks, beer, or mineral water. The technique commonly used to manufacture bottles from PET (ie. to convert the PET into a predetermined shape from a raw material stage) generally involves a two stage process. In the first stage granules of the PET are injection moulded to make a preform. In the second stage the preform is blow moulded to the desired shape.

The softening point of PET is high. Thus, a typical temperature needed for processing of PET is in the region of 260° C. to 285° C. A recognised problem in the industry is that, under the high temperatures and shear conditions needed for injection moulding to make a preform and for blow moulding of the preform to make a bottle, PET tends to degrade, resulting in the formation of acetaldehyde. The presence of acetaldehyde in the material of the finished bottle is undesirable, particularly when the bottle is to be used for products for human consumption, because the acetaldehyde can migrate from the walls of the package or bottle into its contents, whereupon it adversely affects the flavour and fragrance properties of the comestible product. Whilst formation of acetaldehyde in bottles made using virgin PET is an on-going problem, acetaldehyde formation may be even higher in bottles made using recycled PET (rPET) due to the PET having multiple heat histories.

Although the migration of acetaldehyde from a PET bottle into a flavoured beverage is undesirable, a trace of acetaldehyde can often be tolerated because the taste and fragrance of the drink are not usually noticeably affected. However, the presence of even minute amounts of acetaldehyde in either a carbonated or non-carbonated drink, such as mineral water, tends to impart a most undesirable adverse taste and odour to the drink.

It is known to add acetaldehyde scavengers to PET prior to or during melt-processing to scavenge acetaldehyde which may be produced by degradation of the PET. However, there are various competing requirements associated with selection and use of acetaldehyde scavengers. For example, the weight of acetaldehyde scavenger incorporated into the PET needs to be sufficiently high to scavenge a high amount of acetaldehyde. However, higher levels of additives incorporated into PET can be detrimental to optical properties of the PET. For example, high levels of additives may detrimentally impact L*(i.e. reduce L*), a* or b*, each of which is undesirable, particularly when the PET is used for mineral water bottles where aesthetics are particularly important. Additionally, it is important for an acetaldehyde scavenger not itself to migrate significantly from the PET, since this can undesirably enter a beverage contained in a bottle made from the PET.

WO2017/033117A (Colormatrix) discloses a method of decreasing aldehyde content in a polymeric material, for example polyethylene terephthalate. The document specifically exemplifies a range of acetaldehyde scavengers and assesses properties of the scavengers in examples 6 to 13. However, even the scavenger which exhibits the lowest level of migration (example 6) still exhibits relatively high migration and optical properties of bottles incorporating that scavenger could be improved.

The present invention is based on the discovery of aldehyde scavengers which are improved in relation to the scavengers described in WO2017/033117A.

Aldehyde, in particularly, formaldehyde may also be produced during manufacture, processing and/or use of polyoxymethylene (POM) and other polymers. Aldehyde scavengers may be used to scavenge the formaldehyde.

It is an object of the present invention to address problems associated with aldehyde for example formaldehyde or acetaldehyde production in polymeric materials.

It is an object of the present invention to provide aldehyde scavengers which may be improved compared to scavengers described in WO2017/033117A

According to a first aspect of the invention, there is provided a formulation for decreasing aldehyde content, for example in a polymeric material, the formulation including a compound XX which includes at least three moieties of formula

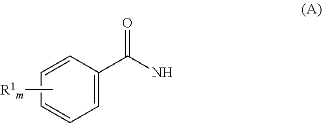

wherein each moiety (A) includes an amine moiety (—NH₂) bonded ortho or meta to the amide moiety (—CONH);
wherein each R¹ independently represents a substituent and m is an integer from 0 to 4; and
wherein the three moieties (A) are bonded, via their respective amide nitrogen atoms, to respective carbon atoms of a Main Fragment, wherein the Main Fragment includes carbon and hydrogen atoms only and is saturated.

It has been found that compounds XX exhibit an advantageous compromise in providing high levels of aldehyde scavenging at acceptable addition rates in a polymeric material, for example in polyester, whilst not significantly impacting optical properties (e.g. L*, a* and/or b*), and, advantageously, exhibit a relatively low level of migration from the polymer. Compounds XX are surprisingly advantageous over compounds disclosed in WO2017/033117A.

One or each R¹ may be selected from a halogen atom, or an optionally-substituted hydrocarbon, alkoxy, amine, amide, phenol or carboxylic acid, group. An optionally-substituted hydrocarbon may be substituted by one or more halogen atoms or by alkoxy, amine, amide, phenol or carboxylic acid, groups. An optionally-substituted hydrocarbon is preferably unsubstituted.

One or each R¹ may be an optionally-substituted, preferably an unsubstituted, alkyl group, for example an optionally-substituted, preferably an unsubstituted, C_{1-20,} for example C_{1-10,} alkyl group. R¹ may be arranged to improve the compatibility of compound XX in the polymeric material in which it may be incorporated, for example by virtue of R¹ including relevant functional groups to improve compatibility.

One or each m may be 0 or 1. Preferably, each m=0. That is, other than the amine and amide moieties, each moiety (A) is unsubstituted.

Preferably, in compound XX, at least one moiety (A) includes an amine moiety (—NH₂) bonded ortho to the amide moiety (—CONH). Preferably in each moiety (A) in compound XX, the amine moiety is bonded ortho to the amide moiety. Preferably, in this case, m=0.

Preferably, said Main Fragment does not include any cyclic or aromatic moiety. Preferably said Main Fragment comprises a linear or branched chain.

Said Main Fragment may include 3 to 20 carbon atoms. Preferably, it includes 5 to 15 carbon atoms, more preferably 7 to 12 carbon atoms and, especially, 8 to 10 carbon atoms. When the number of carbon atoms is n, the number of hydrogen atoms may be equal to 2n-1. Preferably, said Main Fragment includes 5 to 39 hydrogen atoms. Preferably, it includes 9 to 29 hydrogen atoms, more preferably 13 to 23 hydrogen atoms and, especially, 15 to 19 hydrogen atoms.

In a preferred embodiment, said Main Fragment is a C₉H₁₇ moiety.

Said Main Fragment may include a linear chain which includes 6 to 14 carbon atoms, preferably 6 to 10 carbon atoms. The linear chain may include a branch point to which a chain which includes 1 to 4 carbon atoms is attached.

Said Main Fragment may be of general formula

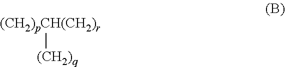

wherein p, q and r are integers, suitably in the range 1 to 10, preferably 1 to 5. Preferably, p is in the range 2 to 4, q is in the range 1 to 3 and r is in the range 2 to 6.

Preferably, the sum of integers p, q and r is at least 4, preferably at least 6, more preferably at least 7. Said sum may be less than 20, preferably less than 15, more preferably less than 10.

In compound XX, preferably the nitrogen atoms of the amide moieties (-CONH) are spaced apart by at least 2, preferably at least 4, carbon atoms; and suitably by no more than 10, for example no more than 7 carbon atoms.

Said compound XX may be of formula

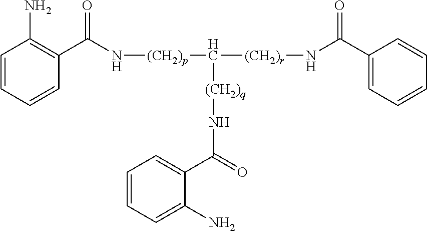

wherein p, q and r are as described above.

Said compound XX is preferably

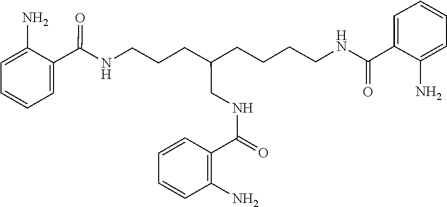 [SMILES: Nc1ccccc1C(=O)NCCCCC(CCCNC(=O)c1ccccc1N)CNC(=O)c1ccccc1N]

Said formulation may include at least 50wt % of said carrier, preferably at least 60wt %, more preferably at least 70wt %, especially at least 75wt %. Said formulation may include less than 80wt % of said carrier.

Said formulation may include 50-95wt % of a carrier, 5-50wt % of said compound XX and 0-30wt % of other additives. Said other additives may be selected from colourants, antioxidants, thickeners, process stabilizers, UV additives and reheat additives. In one preferred embodiment, said formulation includes 0.5 to 10wt % of one or more colourants, for example, at least one blue colourant. In another embodiment, said formulation includes 0.5 to 10wt % of one or more reheat additives, for example titanium nitride or tungsten oxide (especially the latter) as described in WO2016/063013, the content of which is incorporated herein by reference insofar as it relates to titanium nitride and tungsten oxide. In a preferred embodiment, said formulation includes at least some colourant, for example a blue colourant.

A said colourant described herein may be a dye or pigment.

Preferably, in said formulation, the sum of the wt % of carrier(s) and compound XX is at least 80wt %, at least 90wt % or at least 95wt %.

Said formulation may be a solid masterbatch or a liquid formulation. When it is a solid masterbatch, it may comprise 60-95wt % of thermoplastic polymer. Said thermoplastic polymer may be selected from polyesters, polyoxymethylenes (POMs), polyolefins, polyketones and polyurethanes, Said thermoplastic polymer is preferably compatible with and/or includes functional groups which are the same as functional groups in the polymeric material which are to be treated to decrease aldehyde content as described herein.

Said formulation may include 10-40wt % of compound XX and 60-90wt % of thermoplastic polymer.

A solid masterbatch may include up to 60wt % of colourant. A said colourant may be a dye or pigment. A solid masterbatch may include 0 to 10wt %, preferably 0.5 to 10wt %, of one or more colourants, for example one or more inorganic pigments.

When said formulation is a liquid formulation, said formulation may comprise 50 to 90wt % (for example 50 to 80wt %) of liquid carrier and 10 to 50wt % (for example 20 to 50wt %) of compound XX. Said liquid carrier may be a liquid at 25° C. and atmospheric pressure. A carrier is suitably such that it has good solubility in the polymeric material into which it is to be added. It may comprise an oil (e.g. vegetable or mineral oil) or a glycol. A polymeric material-compatible organic liquid carrier (especially wherein said polymeric material is a polyester) may be an oil-based vehicle. Examples of such vehicles are the materials sold as Clearslip™ 2, Clearslip™ 3 & Process Aid-1 by ColorMatrix Europe Ltd, of Units 9-11 Unity Grove, Knowsley Business Park, Merseyside, L34 9GT.

When said formulation is a solid formulation (ie masterbatch), a polymer resin may be used as a carrier. Such a carrier could be a polyester, polyacetal (POM), TPE, polyvinyl butyral (PVB), polyolefin or wax.

Certain compounds of formula XX are believed to be novel. Thus, in a second aspect, there is provided a novel compound of formula XX which includes at least three moieties of formula

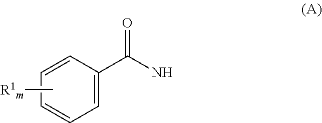

wherein each moiety (A) includes an amine moiety (—NH₂) bonded ortho or meta to the amide moiety (—CONH);
wherein each R¹ independently represents a substituent and m is an integer from 0 to 4; and
   wherein the three moieties (A) are bonded, via their respective amide nitrogen atoms, to respective carbon atoms of a Main Fragment, wherein the Main Fragment includes carbon and hydrogen atoms only and is saturated.

Preferably, said novel compound is

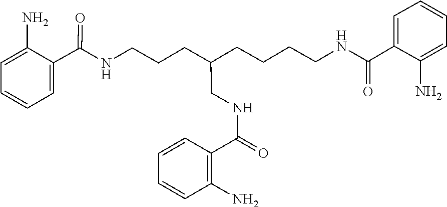 [SMILES: Nc1ccccc1C(=O)NCCCCC(CCCNC(=O)c1ccccc1N)CNC(=O)c1ccccc1N]

According to a third aspect of the invention, there is provided a method of decreasing aldehyde content in a polymeric material, the method comprising the step of contacting the polymeric material, or monomers, oligomers or pre-polymers involved in the preparation of said polymeric material, with a compound XX as described in the first and/or second aspects.

The polymeric material contacted in the method may be any polymeric material which may incorporate an aldehyde in need of being scavenged or otherwise decreased. It may comprise a polyester (especially a poly (ethylene terephthalate)), a polyurethane, a polyoxymethylene (POM), a polyketone or a polyolefin. Preferably, it comprises a polyester, (especially a poly(ethylene terephthalate)). The polymeric material may comprise virgin material or recycled material. The latter may be particularly relevant to HDPE and/or PP.

A reference herein to “ppm” refers to “parts per million” by weight.

Methods for measurement of acetaldehyde in industrially injection-moulded polyethylene terephthalate preforms have been described by FI Villian et al., Journal of Polymer Science, Vol. 52, 55-60 (1994).

Said contacting step may be carried out with the polymeric material in a molten state. Alternatively, said compound may be added to solid polymeric material, suitably at a temperature below the melting point of the polymeric material so the polymeric material is not in a fluid and/or molten state. In one, less preferred, embodiment, compound XX may be added to monomers, oligomers or pre-polymers involved in the preparation of said polymeric material. This may be particularly relevant to processes relating to polyoxymethylenes (POMs), polyvinyl butyral (PVB), polyolefins and polyurethanes.

Advantageously, use of the method may lead to environmental improvements, lower global emissions and sublimation and improved oxygen induction times.

Prior to said contacting step, said polymeric material is preferably selected, suitably when in a solid state as aforesaid. Said selected polymeric material is suitably present substantially in the absence of monomers used in preparation of the polymeric material. Said selected polymeric material is preferably in a state in which it is isolated from a reaction mixture in which it may have been formed. It is preferably an isolated polymeric material. The method may include the step of drying the polymeric material prior to said contacting step. Said selected polymeric material is preferably in a particulate form, for example in the form of pellets, granules or flakes.

The amount of compound XX contacted with said polymeric material may be chosen based upon the level of performance required in polymeric material. In a preferred embodiment the total ppm (based on the weight of said polymeric material) of compound XX contacted with said polymeric material is suitably at least 100 ppm, preferably 200 ppm, more preferably at least 450 ppm. It may be less than 2000 ppm or less than 1000 ppm.

Said compound XX may be a part of a formulation as described according to the first aspect. In a preferred embodiment, compound XX is associated with, for example mixed with, an organic liquid carrier, which is compatible with said polymeric material. Typical carriers include hydrocarbons, hydrocarbon mixtures, alcohols, esters, polyethers and mixtures of two or more thereof.

A polymeric material-compatible organic liquid carrier (especially wherein said polymeric material is a polyester) may be an oil-based vehicle. Examples of such vehicles are the materials sold as Clearslip™ 2, Clearslip™ 3 & Process Aid-1 by ColorMatrix Europe Ltd, of Units 9-11 Unity Grove, Knowsley Business Park, Merseyside, L34 9GT.

When said polymeric material is a polyester, as is preferred, said polyester is preferably a polyethylene terephthalate which term, in the context of the present specification, is intended to encompass co-polyethylene terephthalates. Co-polyethylene terephthalates of polyethylene terephthalate may contain repeat units from at least 85 mole % terephthalic acid and at least 85 mole % of ethylene glycol. Dicarboxylic acids which can be included, along with terephthalic acid, are exemplified by phthalic acid, isophthalic acid, naphthalene-2,6-dicarboxylic acid, cyclohexanedicarboxylic acid, cyclohexanediacetic acid, diphenyl-4,4′-dicarboxylic acid, succinic acid, glutaric acid, adipic acid, azelaic acid and sebacic acid. Other diols which may be incorporated in the co-polyethylene terephthalates, in addition to ethylene glycol, include diethylene glycol, triethylene glycol, 1,4-cyclohexanedimethanol, propane-1,3-diol, butane-1,4-diol, pentane-1,5-diol, hexane-1,6-diol, 3-methylpentane-2,4-diol, 2-methyl pentane-1,4-diol, 2,2,4-trimethylpentane-1,3-diol, 2-ethylhexane-1,3-diol, 2,2-diethylpropane-1,3-diol, hexane-1,3-diol, 1,4-di (hydroxyethoxy)-benzene, 2,2-bis-(4-hydroxycyclohexyl)-propane, 2,4-dihydroxy-1,1,3,3-tetramethyl-cyclobutane, 2,2-bis-(3-hydroxyethoxyphenyl)-propane, and 2,2-bis-(4-hydroxypropoxyphenyl)-propane. In a preferred embodiment said polyethylene terephthalate has less than 10mole %, more preferably less than 6mole % especially less than 2 mole % comonomer substitution. Preferably, said co-polyethylene terephthalate does not comprise co-polyethylene terephthalate; it suitably comprises substantially a homopolymer produced by esterification or transesterification of terephthalic acid or dimethyl terephthalate and ethylene glycol to produce bis (2-hydroxyethyl) terephthalate which is then subjected to polycondensation at high temperatures in vacuum in the presence of a catalyst.

As used herein the term “IV” refers to the Inherent Viscosity of the polymeric material. It may be determined on a solution of 0.5 g of polymer dissolved in 100 ml of a mixture of phenol (60% by volume) and tetrachloroethane (40% by volume).

When said polymeric material is a polyester, the IV of the polyester at the time of contact with said compound XX is preferably greater than 0.5 dL/g, more preferably greater than 0.65 dL/g.

When said polymeric material is a polyester, the polyester may be specifically adapted for use in extrusion blow moulding (EBM). Such adaptations are known to those skilled in the art and include increasing the amount of co-monomers, altering IV and structure.

Said polymeric material is preferably a part of and/or defines an article, for example, a shaped article. Said article may be selected from a preform, a container, a bottle, a cup, a tray, a thermoformed sheet, or other desirable shape.

After contact of said polymeric material with compound XX, said polymeric material may include 50-1000 ppm of compound XX, preferably 100 to 700 ppm or 150 to 600 ppm of compound XX based on the amount of said polymeric material, for example polyester.

According to a fourth aspect of the invention, there is provided a method of making an article, for example a shaped article, from a polymeric material, the method comprising
(a) selecting a compound XX as described in the first aspect and/or second aspects;
(b) contacting the polymeric material with said compound XX; and
(c) forming said polymeric material into an article, for example a shaped article.

Preferably, step (b) is carried out with the polymeric material not in a fluid, for example molten, state. Thereafter, in step (c), the polymeric material is suitably melt-processed to define said article.

Said article may be defined by any process known in the art. For example, said process may comprise injection molding, blow molding or thermoforming. For example, injection molding may be used to form preforms used to blow bottles, food/beverage containers, trays or other desirable shapes. Also, said process may comprise production of a sheet which may subsequently be thermoformed to define an article, for example a receptacle such as a cup or tray. Alternatively, molten polymeric material may be used in extrusion blow molding operations to provide bottles, food containers and the like. Molten polymeric material melt may similarly be fed to an extruder to produce films, sheet, profiles, pipe and the like.

Preferably, said article comprises a container or preform for a container, preferably made from a polyester as described. More preferably, said shaped article comprises a preform, for example for a bottle, such as a beverage bottle.

Said article may include one or more colourants, for example, at least one blue colourant. After contact of said polymeric material with compound XX, said polymeric material may include 1-1000 ppm of a colourant (eg a blue colourant) and, preferably, includes 5-500 ppm of a colourant (eg a blue colourant), the aforementioned ppm being based on the amount of said polymeric material, for example polyester. A said colourant described herein may be a dye or pigment.

Said article may include one or more reheat additives, for example titanium nitride or tungsten oxide (especially the latter) as described in WO2016/063013, the content of which is incorporated herein by reference insofar as it relates to titanium nitride and tungsten oxide. After contact of said polymeric material with compound XX, said polymeric material may include 1-1000 ppm of a reheat additives (eg titanium nitride or tungsten oxide), and, preferably, includes 5-500 ppm of a reheat additives (eg titanium nitride or tungsten oxide), the aforementioned ppm being based on the amount of said polymeric material, for example polyester.

Preferably, in said shaped article, the sum of the wt % of polymeric material(s), for example the amount of polyester, and the amounts of compound XX is at least 90wt %, at least 95wt % or at least 98wt %.

According to a fifth aspect of the invention, there is provided a polymeric material, for example polyester, having a reduced level of aldehyde, for example acetaldehyde, said polymeric material, for example polyester, incorporating a compound XX according to the first and/or second aspects or a product of a reaction between compound XX and aldehyde, for example acetaldehyde.

A product of a reaction between compound XX and aldehyde, for example acetaldehyde, suitably includes a fragment derived from moiety (A) described in the first aspect fragment as follows:

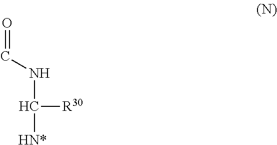

wherein R³⁰ refers to a residue of the aldehyde and is suitably a methyl group when said aldehyde is acetaldehyde.

A product of said reaction preferably includes a moiety

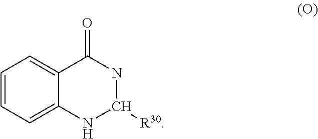

The nitrogen atom adjacent the carbonyl group is suitably bonded to a Main Fragment as described according to the first aspect.

Any aspect of any invention described herein may be combined with any other aspect of any invention described herein mutatis mutandis.

Specific embodiments of the invention will now be described by way of example.

The following materials are referred to hereinafter:

JEFFAMINE (Trade Mark) T-403 polyetheramine of general structure shown below

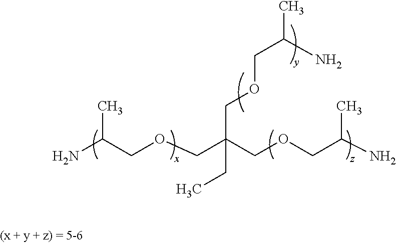

The material is a trifunctional primary amine having molecular weight of approximately 440 Da, when measured by GPC. Its amine groups are located on secondary carbon atoms at the ends of aliphatic polyether chains.

Tris(2-aminoethyl) amine, obtainable from Sigma-Aldrich. It has a boiling point of 114° C. and a structure

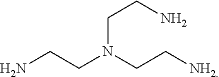

C93 PET—refers to a widely used bottle grade PET from Equipolymers.

In general terms, an aldehyde scavenger is contacted and mixed with polyester, especially PET, and the combination (together with any other additives) is injection moulded to produce a container preform. Preforms are well known. They suitably have a test-tube like body and a threaded neck adjacent an open end, there being a capping flange associated with the neck. Preforms are arranged to be blow moulded to form a container, for example a beverage container that may be closed by a cap which releasably engages a threaded neck.

Aldehyde scavengers described may be solids or liquids. When they are solids, they may be provided as dispersions in a carrier, for example a mineral oil or other carrier which is compatible with the polyester into which the scavenger is to be mixed. When they are liquids, the liquid may be used directly or could be diluted by a carrier as aforesaid. In some embodiment, a carrier for an acetaldehyde scavenger may be solid at 25° C.

Acetaldehyde scavengers may be made as described in Examples 1 to 3, preforms may be made as described in Example 4 and the scavengers assessed as described in subsequent examples.

### Example 1—Preparation of Comparative Acetaldehyde Scavenger Based on Jeffamine™ (Referred to as “Scavenger C1”).

Jeffamine T-403 was reacted with_isatoic anhydride to produce scavenger C1 detailed below, the amount of isatoic anhydride being selected to derivatise all primary amine functional groups of the Jeffamine.

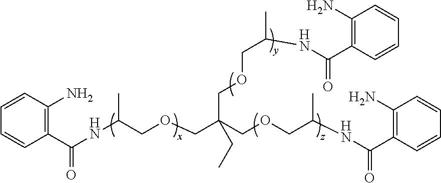

The scavenger is identical to Example 7 in WO2017/033117.

### Example 2-Preparation Of Comparative Acetaldehyde Scavenger Based On Tris(2-Aminoethyl)Amine (Referred to as “Scavenger C2”).

Tris(2-aminoethyl) amine was reacted with isatoic anhydride to produce scavenger C2 detailed below, the amount of isatoic anhydride being selected to derivatise all primary amine functional groups of the Jeffamine.

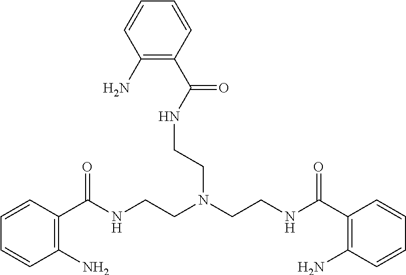 [SMILES: Nc1ccccc1C(=O)NCCN(CCNC(=O)c1ccccc1N)CCNC(=O)c1ccccc1N]

The scavenger is identical to Example 6 in WO2017/033117 and had a melting point of 146-148° C.

### Example 3-Preparation of Acetaldehyde Scavenger N,N′-(2-(4-(2-Aminobenzamido)Butyl)Pentane-1,5-Diyl)Bis(2-Aminobenzamide) (Referred to as “Scavenger EG3”).

Scavenger EG3 has the following structure.

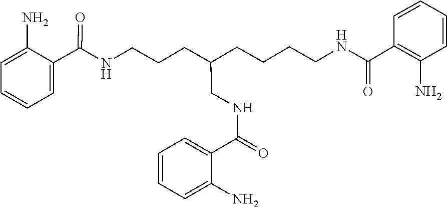 [SMILES: Nc1ccccc1C(=O)NCCCCC(CCCNC(=O)c1ccccc1N)CNC(=O)c1ccccc1N]

It was made as follows:

2H-benzo[d][1,3] oxazine-2,4(1H)-dione (98.84 g, 3.5 Eq, 605.9 mmol) was dissolved in dimethylformamide (500 ml) at room temperature. To the reaction mixture was drop-wise added a solution of 4-(aminomethyl)octane-1,8-diamine (30.00 g, 1 Eq, 173.1 mmol) in dimethylformamide (250 ml). The reaction mixture was stirred at room temperature overnight until full conversion was evidenced by LC-MS. The dimethylformamide was removed under reduced pressure yielding a dark brown oil. To this was added water (1 I), ammonium hydroxide (25%, 50 ml) and the product extracted with dichloromethane. The dichloromethane was removed under reduced pressure and the product recrystallized in a mixture of methanol and acetonitrile. The solids were collected by filtration and dried to yield N,N′-(2-(4-(2-aminobenzamido)butyl)pentane-1,5-diyl)bis(2-aminobenzamide) (51.0 g, 55.5% yield). The structure of the compound was confirmed by NMR and LC-MS and the melting point was 160° C.

### Example 4—General Procedure for Preparation of Preforms

C93 PET resin is dried prior to use using Con-Air (Trade Mark) dryers for at least four hours at 160° C.

A selected amount of scavenger was contacted with hot, dry C93 PET in the presence of 1000ppm (relative to the C93 PET) of an acetic acid ester of monoglycerides made from fully hydrogenated castor oil. The latter is a carrier. Whilst, in practice, a liquid formulation comprising carrier and scavenger would be used, the separate addition of scavenger and carrier described is convenient for the experimental procedure.

The blend was added into the feedthroat of a Husky 160T Injection Molder with the following parameters and 34 g PET preforms were produced.

| Polymer | Equipolymers C93 PET |
|---|---|
| Drying conditions | Motan Drier 160° C., 4 hours |
| Machine name | Husky |
| Mould | 2 cavity, 33 g perform |
| Zone 5 temp. (° C.) | 285 |
| Zone 4 temp. (° C.) | 285 |
| Zone 3 temp. (° C.) | 285 |
| Zone 2 temp. (° C.) | 285 |
| Zone 1 temp. (° C.) | 285 |
| Nozzle temp. (° C.) | 285 |
| Mold temp. (° C.) | 285 |

### Example 5—General Procedure for Determining Acetaldehyde Content of Preform Samples

The acetaldehyde content of samples is determined on preform samples that have been cryo-ground to less than 1 mm. The level of acetaldehyde is determined using a ThermoFisher 22 Scientific Trace 1310 gas chromatograph with a Triplus 500 headspace autosampler and FID detector. Acetaldehyde reductions are calculated on the basis of percentage reduction seen in the acetaldehyde levels of a preform with additives, compared to that with no additives.

### Example 6—Procedure for Measuring Optical Properties

Samples were taken from the side walls of preforms, relevant controls were made and optical properties (i.e. L*, a* and b*) were assessed using a Minolta CM-3700d spectrophotometer in transmission mode fitted with a D65/10° light source.

### Example 7—Procedure for Determining Migration of Acetaldehyde Scavenger from PET

Bottles blown from preforms incorporating selected acetaldehyde scavengers along with relevant controls were filled with 20% ethanol/water (in accordance with standard EU accepted test conditions) and placed in an oven at 60° C. for 10 days. At various times, the water was sampled using LC-MS to determine the level (if any) of migration of acetaldehyde scavengers into the water. Multiple bottles were assessed for each scavenger tested and an average migration value calculated.

### Example 8—Assessment of Acetaldehyde Scavenging Ability of Selected Materials

Using the general procedure described in Example 4, preforms were made using a range of compounds described and the acetaldehyde scavenging ability of the_compounds assessed as described in Example 5. Results are provided below. Note that, based on other experiments (not reported), the loading of each scavenger was selected based on the level required to achieve a comparable level of acetaldehyde reduction to that when 500 ppm of anthranilamide, a commercially available acetaldehyde scavenger, is used.

Results relating to the acetaldehyde scavenging ability of the acetaldehyde scavengers of Examples 1 to 3, anthranilamide as a control (consisting of C93 PET) are provided in the table below:

| | Loading | |
|---|---|---|
| Identity of scavenger | (ppm) | Acetaldehyde reduction (%) |
| Control - no scavenger | 0 | n/a |
| Anthranilamide | 500 | 69 |
| Scavenger C1 from Example 1 | 980 | 43 |
| Scavenger C2 from Example 2 | 600 | 73 |
| Scavenger EG3 from Example 3 | 550 | 70 |

n/a means “not applicable”.

### Example 9—Assessment of Optical Properties of Selected Materials

The preforms referred to in Example 8 were assessed as described in Example 6. Results relating to optical properties (L*, a*, b*) are provided in the table below:

| | Loading | | | |
|---|---|---|---|---|
| Identity of scavenger | (ppm) | L*(D65) | a*(D65) | b*(D65) |
| Control - no scavenger | 0 | 86.15 | −0.42 | 1.57 |
| Anthranilamide | 500 | 85.43 | −2.18 | 7.78 |
| Scavenger C1 from Example 1 | 980 | 84.46 | −5.31 | 23.09 |
| Scavenger C2 from Example 2 | 600 | 83.32 | −3.22 | 12.40 |
| Scavenger EG3 from Example 3 | 550 | 84.84 | −3.62 | 12.11 |

### Example 10—Assessment of Migration of Acetaldehyde Scavengers

The preforms referred to in Example 8 were assessed as described in Example 7. Results relating to the level of migration in “parts per billion” (ppb) by weight are provided in the table below:

| | Loading | Migration |
|---|---|---|
| Identity of scavenger | (ppm) | (ppb) |
| Control - no scavenger | 0 | n/a |
| Anthranilamide | 500 | 116 |
| Scavenger C1 from Example 1 | 980 | 2 |
| Scavenger C2 from Example 2 | 600 | 0.6 |
| Scavenger EG3 from Example 3 | 550 | 0.2 |

n/a means “not applicable”.

### Example 11-Calculations to Compare Efficacy of Acetaldehyde Scavengers

Based on the preceding examples and results and to illustrate advantageous characteristics of the scavenger EG3, the following calculation were undertaken:
(i) The amount of acetaldehyde scavenger to treat 1 Kg of PET. For this it is desirable for the amount to be relatively low whilst achieving a high level of acetaldehyde reduction.
(ii) Molecular weight (MW) of scavenger.
(iii) The mmol of acetaldehyde scavenger used to achieve the specified level of scavenging. A low value is preferred.
(iv) The mmol of nitrogen-containing active centres to achieve the specified level of scavenging.
(v) The mmol of nitrogen-containing active centres divided by the % acetaldehyde reduction to achieve the specified level of scavenging. A low value is preferred.
(vi) The mmol of nitrogen-containing active centres divided by the migration in ppb acetaldehyde reduction to achieve the specified level of scavenging. A high value is preferred.

| | Characteristic reference no. | | | | | | |
|---|---|---|---|---|---|---|---|
| Identity of | Loading | (i) g/kg | | (iii) | (iv) | (v) | (vi) |
| scavenger | (ppm) | PET | (ii) | mmol | mmol | mmol/% | mmol/ppb |
| Control - no | 0 | n/a | n/a | n/a | n/a | n/a | n/a |
| scavenger | | | | | | | |
| Anthranilamide | 500 | 0.5 | 136.15 | 3.672 | 1.224 | 0.0177 | 0.01 |
| Scavenger C1 | 980 | 0.98 | 797.00 | 1.230 | 1.230 | 0.0286 | 0.61 |
| from Example | | | | | | | |
| 1 | | | | | | | |
| Scavenger C2 | 600 | 0.6 | 503.60 | 1.191 | 1.191 | 0.0163 | 1.99 |
| from Example | | | | | | | |
| 2 | | | | | | | |
| Scavenger | 550 | 0.55 | 530.66 | 1.036 | 1.036 | 0.0148 | 5.18 |
| EG3 from | | | | | | | |
| Example 3 | | | | | | | |

### Discussion

It will be noted from the above that Scavenger EG3 has very low migration as per (vi), has best in class efficacy (see (iii) and (v)) and has higher L* and lower b* compared to other low migration scavengers.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A formulation for decreasing aldehyde content, for example in a polymeric material, the formulation including a compound XX which includes at least three moieties of formula 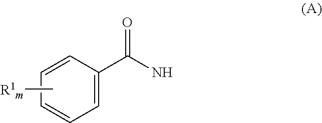
wherein each moiety (A) includes an amine moiety (—NH₂) bonded ortho or meta to the amide moiety (—CONH);
wherein each R¹ independently represents a substituent and m is an integer from 0 to 4; and
wherein the three moieties (A) are bonded, via their respective amide nitrogen atoms, to respective carbon atoms of a Main Fragment, wherein the Main Fragment includes carbon and hydrogen atoms only and is saturated.

2. (canceled)

3. A formulation according to claim 1, wherein each m=0.

4. A formulation according to claim 1, wherein, in each moiety (A) in compound XX, the amine moiety is bonded ortho to the amide moiety.

5. A formulation according to claim 1, wherein said Main Fragment does not include any cyclic or aromatic moiety; and/or said Main Fragment comprises a linear or branched chain.

6. A formulation according to claim 1, wherein said Main Fragment includes 3 to 20 carbon atoms.

7. A formulation according to claim 1, wherein said Main Fragment is of general formula 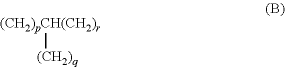
wherein p, q and r are integers in the range 1 to 10.

8. A formulation according to claim 7, wherein p is in the range 2 to 4, q is in the range 1 to 3 and r is in the range 2 to 6.

9. A formulation according to claim 7, wherein said compound XX is of formula

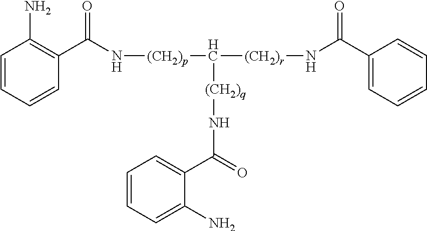

10. A formulation according to claim 1, wherein said compound XX is

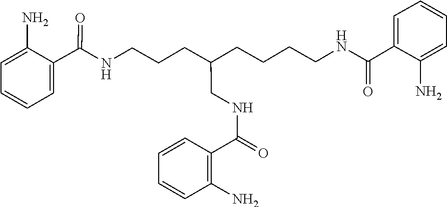 [SMILES: Nc1ccccc1C(=O)NCCCCC(CCCNC(=O)c1ccccc1N)CNC(=O)c1ccccc1N]

11. A formulation according to claim 1, wherein said formulation includes 50-95 wt % of a carrier, 5-50 wt % of said compound XX and 0-30 wt % of other additives.

12. A formulation according to claim 1, wherein said formulation includes 0.5 to 10 wt % of one or more colourants.

13. (canceled)

14. A formulation according to claim 1, wherein said formulation is a solid masterbatch comprising 60-95 wt % of thermoplastic polymer selected from polyesters, polyoxymethylenes (POMs), polyolefins, polyketone, polyvinyl butyral (PVB) and polyurethanes; and 0.5 to 10 wt %, of one or more colourants.

15. A formulation according to claims 1, wherein said formulation is a liquid formulation comprising 50 to 90 wt % of liquid carrier and 10 to 50 wt %.

16. (canceled)

17. (canceled)

18. A method of decreasing aldehyde content in a polymeric material, the method comprising the step of contacting the polymeric material, or monomers, oligomers or pre-polymers involved in the preparation of said polymeric material, with a compound XX as described in claim 1.

19. (canceled)

20. A method according to claim 18, wherein the polymeric material contacted in the method comprises a polyester, and optionally the total ppm (based on the weight of said polymeric material) of compound XX contacted with said polymeric material is at least 100 ppm.

21. (canceled)

22. (canceled)

23. A method according to claim 18, wherein said polymeric material is a part of shaped article which is selected from a preform, a container, a bottle, a cup, a tray and a thermoformed sheet.

24. (canceled)

25. (canceled)

26. A polymeric material having a reduced level of aldehyde, for said polymeric material incorporating a compound XX according to claim 1, or a product of a reaction between compound XX according to claim 1 and aldehyde.

27. A polymeric material according to claim 26, wherein said polymeric material incorporates a product of a reaction between compound XX and aldehyde, wherein said product of said reaction includes a moiety

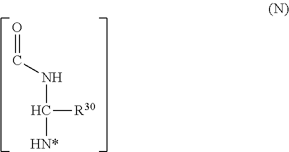

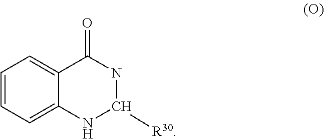

28. (canceled)

29. A formulation according to claim 1, wherein:
said formulation includes at least 50wt % of a carrier which is an oil; or
said formulation is a solid masterbatch comprising 60-95 wt % of thermoplastic polymer; or
said formulation includes 0.5 to 10wt % of one or more colourants.

30. A formulation according to claim 1, wherein said formulation includes at least 50wt % of a carrier which is an oil.
